(19) United States
(12) Patent Application Publication
(10) Pub. No.: US 2025/0354189 A1
(43) Pub. Date: Nov. 20, 2025

(54) NATTO OLIGOPEPTIDES, PREPARATION METHOD THEREOF AND APPLICATION IN THROMBOLYSIS AND BLOOD PRESSUREREDUCTION

(71) Applicant: DALIAN BLUE PEPTIDE TECHNOLOGY RESEARCH & DEVELOPMENT CO., LTD., DALIAN (CN)

(72) Inventors: WEI GAO, DALIAN (CN); YANSHENG ZHANG, DAILIAN (CN); ZUZHE WANG, DALIAN (CN); WEIYANG BAO, DALIAN (CN); YANNA YUAN, DALIAN (CN)

(73) Assignee: DALIAN BLUE PEPTIDE TECHNOLOGY RESEARCH & DEVELOPMENT CO., LTD., DALIAN (CN)

(21) Appl. No.: 19/077,122

(22) Filed: Mar. 12, 2025

(30) Foreign Application Priority Data
May 15, 2024 (CN) 202410603422.1

(51) Int. Cl. C12P 21/06; C07K 1/18; C12N 9/54

(52) U.S. Cl. CPC C12P 21/06; C07K 1/18; C12N 9/54; C12Y 304/21062

(57) ABSTRACT

This invention pertains to bioactive peptide preparation, addressing the challenge of obtaining natto-derived polypeptides with antithrombotic effects. By analyzing a bioactive peptide database, key characteristics of antithrombotic peptides are identified: molecular weight <2000 Da, N-terminal glycine (G), C-terminal lysine (K) or arginine (R), and net charge ±2. The distribution of amino acids preceding glycine and arginine in natto hydrolysates is statistically analyzed. Based on frequency rankings, specific peptide bonds are selected as cleavage sites, and composite proteases (e.g., trypsin-chymotrypsin blends) enzymatically hydrolyze these sites. Low-molecular-weight peptides (±2 net charge) are purified via ultrafiltration and ion-exchange chromatography. The method preserves nattokinase's thrombolytic activity while enriching free L-arginine, synergistically enhancing antithrombotic and hypotensive effects. Applications include mitigating thrombotic cardiovascular diseases through dual thrombolytic and vasoregulatory mechanisms.

### TECHNICAL FIELD

The present invention pertains to the field of bioactive peptide preparation, specifically relating to a method for preparing natto oligopeptides and their application in thrombolysis and blood pressure reduction.

### BACKGROUND

Thrombotic diseases, as typical cardiovascular diseases, pose a serious threat to human life and health, characterized by high incidence, disability, and mortality rates. Their incidence and mortality rates rank among the top of various diseases, causing great suffering to patients and significant impacts on their families. Additionally, a range of cardiovascular diseases, including thrombosis, can be triggered by persistent hypertension. Currently, there are several thrombolytic agents used in clinical practice for thrombolytic therapy, such as streptokinase, urokinase, staphylokinase, single-chain urokinase, and tissue-type plasminogen activator. These thrombolytic agents have high specificity for thrombolysis but are also prone to causing allergic reactions and severe bleeding.

Extensive research has shown that natto has various health benefits, including preventing osteoporosis, promoting blood coagulation, lowering blood pressure, and improving blood sugar levels. The health benefits of natto are closely related to the changes in the basic components of soybeans during the production process. Proteins, fats, starches, and isoflavones in soybeans undergo complex biochemical reactions under the action of Bacillus subtilis natto, retaining original active components while generating new active substances such as nattokinase (NK), antioxidant peptides, and antimicrobial peptides. Related studies have shown that nattokinase has certain effects on anti-platelet aggregation, in vitro thrombolysis, blood pressure reduction, and lipid-lowering. Its thrombolytic mechanism includes the direct hydrolysis of fibrin and plasmin substrates to dissolve blood clots. It converts endogenous kinases into urokinase and degrades plasminogen activator inhibitors, increasing the level of tissue plasminogen activator, thereby dissolving thrombi.

However, research on natto components other than nattokinase, especially bioactive peptides, is still relatively scarce. Yahui Song discovered that natto contains peptides with high ACE inhibitory activity (RBPs) through fermentation. Studies on their properties found that RBPs significantly reduce the incidence of spontaneous hypertension in rats and have protective effects on the kidneys, thoracic aorta, and heart. Kousuke Sato identified the in vitro DPPIV inhibitory activity of natto isolated peptides. These studies indicate that natto contains various bioactive peptide components. The Chinese invention patent application CN202310653994.6 discloses a method for preparing natto and natto bioactive peptides by inoculating composite bacteria to ferment soybeans, grinding the mature fresh natto into a semi-fluid, drying, and then crushing to obtain natto bioactive peptides. These studies focus on isolating the inherent bioactive peptide components in natto products without further exploring the potential of natto protein/bioactive peptide resources. The Chinese invention patent application CN202311164212.9 discloses a method for preparing natto lipid-lowering peptides and their application, and the Chinese invention patent CN202311164254.2 discloses a method for preparing natto antioxidant bioactive peptides and their beverage. However, these methods do not involve the most notable anti-thrombotic effects of natto, and the acidic or alkaline environment during enzymatic hydrolysis and the high temperature during enzyme deactivation are highly likely to damage the activity of nattokinase. Lee K-A et al. isolated two anti-thrombotic peptide segments, SSGE and DEE, from soybean protein, which have anti-platelet aggregation effects. Although the fermentation of soybean protein by natto bacteria causes some changes in its components, it is reasonable to believe that natto has the potential for anti-thrombotic peptides. Currently, the most common natto product on the market besides natto itself is natto freeze-dried powder, categorized by nattokinase activity. Related research mainly focuses on improving fermentation processes to increase nattokinase content, with little development and utilization of components other than nattokinase. Maximizing the use of natto resources by developing active components other than nattokinase to enhance the improvement of thrombotic cardiovascular and cerebrovascular diseases is of great importance.

### SUMMARY

To address the issue of obtaining functional oligopeptides and further solve the problem of acquiring natto-derived polypeptides with anti-thrombotic effects, in the first aspect, the method for preparing oligopeptides according to some embodiments of the present application includes:

Retrieving and determining the characteristics of peptides related to the functionality of biological objects from a bioactive peptide database, where the characteristics include molecular weight distribution, types of N-terminal amino acid residues, types of C-terminal amino acid residues, and net charge;

Preparing a peptide solution from the biological object and statistically analyzing the types and quantities of amino acids preceding specific N-terminal amino acids in the peptide solution;

Using composite proteases to cleave peptide bonds at specific N-terminal amino acids and/or C-terminal carboxyl-terminal peptide bonds, with the cleavage sites selected based on the identified characteristics;

Filtering the cleaved peptide solution and collecting the peptide solution with a molecular weight distribution that matches the characteristics;

Separating and purifying the peptide solution with the matching molecular weight distribution, and collecting the peptide solution with the specified net charge to obtain the oligopeptides.

In the second aspect, the method for preparing oligopeptides according to some embodiments of the present application includes:

Retrieving and determining the characteristics of peptides related to natto's anti-thrombotic function from a bioactive peptide database, where the determined characteristics include a molecular weight below 2000 Da, N-terminal amino acid residue type as glycine (G), C-terminal amino acid residue type as lysine (K) or arginine (R), and a net charge of ±2;

Preparing a peptide solution from natto and statistically analyzing the types and quantities of amino acids preceding glycine (G) and arginine (R) in the peptide solution;

Sorting the identified peptide bonds based on statistical quantities, and using composite proteases to cleave peptide bonds such as tyrosine-glycine, phenylalanine-glycine, tyrosine-arginine, lysine carboxyl-terminal, and arginine carboxyl-terminal, where the composite proteases consist of trypsin and chymotrypsin in a ratio of 1:(1-3), with enzymatic hydrolysis conditions: temperature 37-45° C., pH 7.5-8.5, and time 3-6 hours;

Filtering the cleaved peptide solution and collecting the peptide solution with a molecular weight below 2000 Da;

Separating and purifying the peptide solution with a molecular weight below 2000 Da, and collecting the peptide solution with a net charge of ±2 to obtain the first oligopeptides.

According to some embodiments of the method for preparing oligopeptides, the method further includes mixing the peptide solution with a net charge of ±2 with a nattokinase component solution to obtain the second oligopeptides, where:

Preparing the nattokinase component solution includes:

Filtering the natto powder solution through an ultrafiltration membrane with a molecular weight cutoff of 29000 Da to obtain the first filtrate;

Filtering the first filtrate through an ultrafiltration membrane with a molecular weight cutoff of 26000 Da to obtain the second filtrate and the second retentate;

Where the second retentate is the nattokinase component solution.

According to some embodiments of the method for preparing oligopeptides, the natto is prepared from soybeans.

According to some embodiments of the method for preparing oligopeptides, the method further includes freeze-drying the first oligopeptides to obtain natto oligopeptide powder.

According to some embodiments of the method for preparing oligopeptides, the method further includes freeze-drying the second oligopeptides to obtain natto oligopeptide powder.

According to some embodiments of the method for preparing oligopeptides, preparing the natto small peptide solution includes adding digestive enzymes to the first filtrate and the second retentate to obtain the natto small peptide solution.

According to some embodiments of the method for preparing oligopeptides, the digestive enzyme is trypsin.

According to some embodiments of the method for preparing oligopeptides, filtering the cleaved peptide solution includes filtering the cleaved peptide solution through a nanofiltration membrane with a molecular weight cutoff of 2000 Da, where the filtrate is the peptide solution with a molecular weight below 2000 Da;

Where separating and purifying the peptide solution with a molecular weight below 2000 Da includes using ion-exchange chromatography for separation and purification.

According to some embodiments of the method for preparing oligopeptides, the ion-exchange chromatography includes a strong cation-exchange column.

According to some embodiments of the method for preparing oligopeptides, the ion-exchange chromatography includes an SP Sepharose High Performance strong cation-exchange column, where after loading, the column is equilibrated with 20 mmol/L PB (pH 6.0), and eluted with a 0-1 M NaCl gradient over 0-40 minutes, collecting the eluate from 11-19 minutes to obtain the peptide solution with a net charge of ±2.

According to some embodiments of the method for preparing oligopeptides, sequencing the peptides in the natto peptide solution, and statistically analyzing the types and quantities of amino acids preceding glycine (G) and arginine (R) based on the sequencing;

Where sorting the statistical quantities includes:

Sorting the types of amino acids preceding glycine (G) based on quantity;

Sorting the types of amino acids preceding arginine (R) based on quantity;

Based on the sorting of amino acids preceding glycine (G), cleaving the peptide bonds of the most or top few abundant amino acids with glycine (G) to obtain peptides with N-terminal glycine (G);

Based on the sorting of amino acids preceding arginine (R), cleaving the peptide bonds of the most or top few abundant amino acids with arginine (R) to obtain peptides with N-terminal arginine (R);

Cleaving lysine carboxyl-terminal peptide bonds to obtain peptides with C-terminal lysine (K);

Cleaving arginine carboxyl-terminal peptide bonds of peptides with non-N-terminal arginine (R) to obtain peptides with C-terminal arginine (R), and cleaving arginine (R) of peptides with N-terminal arginine (R) to obtain free L-arginine.

In the third aspect, the oligopeptides prepared by any of the methods according to some embodiments of the present application.

In the fourth aspect, the use of the oligopeptides according to some embodiments of the present application in the preparation of thrombolytic drugs, antihypertensive drugs, or drugs for simultaneous thrombolysis and antihypertension.

The beneficial effects are as follows:
(1) In the first aspect, the present invention analyzes the physicochemical properties of functional peptide segments through an online database, selecting molecular weight distribution, types of N-terminal and C-terminal amino acid residues, and net charge as characteristics to determine enzyme cleavage sites. By cleaving small peptides with composite proteases at these sites and further filtering and purifying to meet the specified molecular weight and net charge requirements, the invention obtains small peptides with the desired characteristics, enabling them to exhibit targeted functionality.
(2) In specific examples, the present invention analyzes the physicochemical properties of anti-thrombotic peptide segments through an online database, maximizing the release of anti-thrombotic peptides from natto. By combining with nattokinase components, the thrombolytic activity of natto freeze-dried powder is further enhanced. Additionally, the process increases the amount of free L-arginine (L-Arg), which helps dilate blood vessels, regulate blood pressure, and reduce the risk of thrombosis caused by endothelial damage due to high blood pressure, alleviating symptoms of thrombotic cardiovascular diseases such as atherosclerosis.
(3) L-Arg is the sole substrate for nitric oxide (NO) production in all mammals. NO promotes vasodilation, increases blood flow, improves circulation, and helps regulate blood pressure. It also inhibits platelet aggregation and coagulation, reducing the risk of thrombosis. Experimental results from animals and clinical studies show that L-Arg influences platelets, coagulation, and fibrinolysis through the NO pathway. The invention enhances the quantity of specific small peptides through composite enzyme cleavage, achieving targeted functionality. Simultaneously, by considering the role of L-Arg and designing composite enzymes, the invention increases the amount of free L-Arg, providing both thrombolytic and antihypertensive effects, with the latter further reducing the risk of thrombosis.

### BRIEF DESCRIPTION OF DRAWINGS

FIG. 1 is a schematic view showing distribution of amino acid residue types preceding glycine.

FIG. 2 is a schematic view showing distribution of amino acid residue types preceding arginine.

FIG. 3 is a schematic view showing distribution of N-terminal amino acid residue types.

FIG. 4 is a schematic view showing distribution of C-terminal amino acid residue types.

### DETAILED DESCRIPTION

The following examples further illustrate the present invention. It should be noted that the examples do not limit the scope of protection claimed by the present invention.

The present invention provides a method for preparing natto oligopeptides and their application in thrombolysis and blood pressure reduction. The preparation method includes the following steps:

S1: Using natto freeze-dried powder prepared by fermentation with natto bacteria as the raw material, dissolving it in physiological saline, and separating it through an ultrafiltration membrane to obtain a solution containing components with a molecular weight between 26000-29000 Da, referred to as the nattokinase component F1. The remaining components after ultrafiltration separation, which include components with a molecular weight less than 26000 Da and greater than 29000 Da, are referred to as the natto component F2 solution.

S2: Searching the keyword “antithrombotic” in the “Bioactive peptides” module of the BIOPEP database to analyze the characteristics of bioactive peptides with antithrombotic activity, including molecular weight distribution, types of N-terminal amino acid residues, types of C-terminal amino acid residues, net charge, and other basic characteristics. In the present invention, the characteristic information specifically refers to peptides with a molecular weight below 2000 Da, N-terminal amino acid residue type as glycine (G), C-terminal amino acid residue type as lysine (K) or arginine (R), and a net charge of ±2 as potential antithrombotic peptide segments.
S3: Digesting the remaining natto component F2 after isolating the nattokinase component F1 with digestive enzymes such as trypsin for 4 hours to obtain the first natto hydrolysate. The first natto hydrolysate is analyzed by LC-MS/MS and de novo sequencing to determine peptide segments. Based on the above characteristics, the invention aims to obtain oligopeptides with N-terminal amino acid residues as glycine and C-terminal amino acid residues as lysine (K) or arginine (R). The C-terminal amino acid residues as lysine (K) or arginine (R) are obtained by cleaving lysine carboxyl-terminal peptide bonds or arginine carboxyl-terminal peptide bonds, respectively, using enzymes such as trypsin.

To obtain oligopeptides with N-terminal amino acid residues as glycine, the types and quantities of amino acid residues preceding glycine are statistically analyzed. Considering the influence of composite proteases, which can cleave multiple sites, it is difficult to cleave all types of peptide bonds preceding glycine. In the present invention, the most abundant or the top two most abundant types of amino acids preceding glycine are selected for cleavage, ensuring an effective quantity of oligopeptides with N-terminal glycine while reducing the design difficulty of the composite protease.

The invention also statistically analyzes the types and quantities of amino acid residues preceding arginine and designs composite proteases to cleave peptide bonds between these amino acids and arginine simultaneously, obtaining an effective quantity of oligopeptides with N-terminal arginine. Although the invention does not require oligopeptides with N-terminal arginine based on the characteristics in step S2, the cleavage of arginine carboxyl-terminal peptide bonds produces oligopeptides with C-terminal arginine (R). For these oligopeptides, if N-terminal arginine is obtained, the cleavage results in free L-arginine, which helps dilate blood vessels and reduce blood pressure. Therefore, the invention also includes the statistical analysis and cleavage of amino acids preceding arginine.

Based on the above statistical analysis, the cleavage sites are determined, and a composite protease combination is designed. The natto component F2 is enzymatically hydrolyzed under suitable conditions to obtain the second hydrolysate. The composite protease of the invention must respond to the cleavage targets of the above characteristics and achieve the goal of obtaining free L-arginine. The invention uses a combination of trypsin and chymotrypsin to achieve this purpose.

S4: The second hydrolysate is separated through a nanofiltration membrane with a molecular weight cutoff of 2000 Da. The filtrate is collected to obtain the natto hydrolysate component F3 solution.

S5: The natto hydrolysate component F3 solution is further purified by ion-exchange chromatography, and the solution with a net charge of ±2 is collected as the natto hydrolysate component F4 solution. The natto hydrolysate component F4solution has thrombolytic and antihypertensive effects.

S6: The natto hydrolysate component F4 solution is thoroughly mixed with the nattokinase component F1 solution obtained in step S1, which has anti-platelet aggregation, in vitro thrombolysis, blood pressure reduction, and lipid-lowering effects. The mixture is then freeze-dried to obtain natto oligopeptide powder.

In the first aspect, the invention first isolates the nattokinase component F1 to avoid damage to nattokinase during subsequent enzymatic hydrolysis. After preparing the natto hydrolysate component F4 with thrombolytic and antihypertensive effects, the nattokinase component F1 is mixed with the natto hydrolysate component F4 to further enhance the therapeutic effects and increase the quantity of the product for treatment.

In the second aspect, the invention statistically analyzes the peptide sequences of the natto component F2, targeting amino acids preceding glycine, amino acids preceding arginine, lysine carboxyl-terminal, and arginine carboxyl-terminal as cleavage sites. A composite protease formulation is designed for enzymatic hydrolysis to obtain the natto hydrolysate component F3, which contains a high content of free L-arginine with antihypertensive effects and increases the content of peptide segments with N-terminal and C-terminal amino acid residue characteristics of antithrombotic activity.

In the third aspect, the invention further purifies the natto hydrolysate component F4 with a molecular weight below 2000 Da and a net charge of ±2 through ultrafiltration membrane separation and ion-exchange chromatography. Since arginine carries one positive charge, it can be eluted together with component F4 in ion-exchange chromatography. Therefore, the L-arginine obtained by enzymatic hydrolysis does not require modifications in the separation and purification steps.

Example 1: A method for preparing oligopeptides includes the following steps:

S1: Dissolving 100 g of natto freeze-dried powder prepared by fermentation with natto bacteria in 1000 ml of physiological saline. The solution is passed through a 29000 Da ultrafiltration membrane, and the filtrate is collected to obtain components with a molecular weight below 29000 Da. The filtrate is then passed through a 26000 Da ultrafiltration membrane, and the retentate is collected to obtain components with a molecular weight above 26000 Da but below 29000 Da, referred to as the nattokinase component F1. The retentate from the first ultrafiltration and the filtrate from the second ultrafiltration are combined as the natto component F2.

S2: The natto component F2 solution is hydrolyzed with digestive enzymes such as trypsin for 4 hours to obtain the natto hydrolysate (hydrolysis pH 8.5, hydrolysis temperature 50° C.). The purpose of hydrolysis is to break down proteins into small peptides (small molecular peptides, typically composed of 2 to 20 amino acid residues). The hydrolysate is analyzed by LC-MS/MS and de novo sequencing for peptide analysis under the following conditions:
Chromatographic conditions: Column: C18, 3 μm, 75 μm*15 cm; Mobile phase: A: 0.1% formic acid in water; B: 0.1% formic acid in 80% acetonitrile/H₂O; Gradient program: Spray voltage: 2.0 kV; Capillary temperature: 320° C.; RF Lens: 40; Resolution settings: MS1 120,000 @m/z 200, MS2 30,000 @ m/z 200; Precursor ion scan range: m/z 350-1550; Product ion scan range: start from m/z 110; Fragmentation mode: HCD.

The raw data acquired from mass spectrometry were analyzed using the de novo sequencing software PEAKS. The software parameters were set as follows:

Enzyme: Non-specific; Variable modifications: Oxidation (M), Deamidation (N, Q); Peptide mass tolerance: ±10 ppm; Fragment mass tolerance: 0.02 Da.

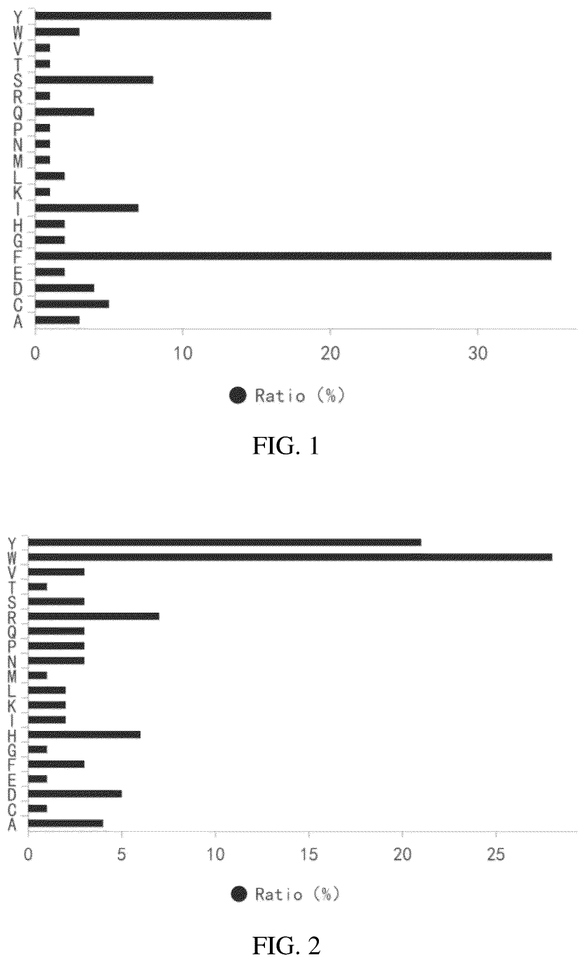

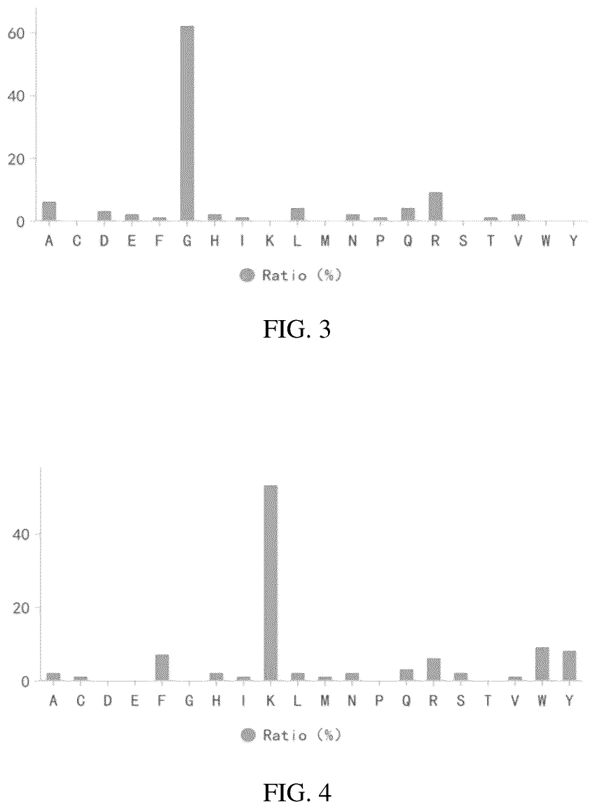

The types and quantities of amino acid residues preceding glycine and arginine were statistically analyzed. The results showed the distribution of amino acid residues preceding glycine and arginine, as illustrated in FIGS. 1 and 2, respectively. Based on this, the peptide bonds of tyrosine-glycine, phenylalanine-glycine, tyrosine-arginine, lysine carboxyl-terminal, and arginine carboxyl-terminal were selected as target cleavage sites for enzymatic hydrolysis of the natto component F2. The hydrolysis conditions were as follows: trypsin: chymotrypsin=1:1-3, enzyme dosage: 1-3%, hydrolysis temperature: 37-45° C., hydrolysis pH: 7.5-8.5, and hydrolysis time: 3-6 hours, resulting in the second hydrolysate.

S3: The second hydrolysate was separated through a nanofiltration membrane with a molecular weight cutoff of 2000 Da, and the filtrate was collected to obtain the natto hydrolysate component F3, which contained oligopeptides with a molecular weight below 2000 Da.

S4: An SP Sepharose High Performance strong cation-exchange column was equilibrated with 20 mmol/L PB (pH 6.0). After loading and re-equilibration, elution was performed with a 0-1 M NaCl gradient over 0-40 minutes, and the eluate collected between 11-19 minutes was designated as the natto hydrolysate component F4.

S5: The natto hydrolysate component F4 was thoroughly mixed with the nattokinase component F1 and freeze-dried to obtain natto oligopeptide powder.

### Experimental Example 1: Determination of L-Arginine Content

The natto freeze-dried powder used in step S1 of Example 1 and the natto oligopeptide powder obtained in step S5 were prepared into aqueous solutions at a concentration of 100 μg/ml. Standard L-arginine solutions were prepared at concentrations of 0.625, 1.25, 2.5, and 5.0 μmol/ml using deionized water. Samples and standards (10 μL each) were derivatized with 20 μL of 6-aminoquinoline-N-hydroxysuccinimidyl carbamate solution, and the free L-arginine content was determined by high-performance liquid chromatography with fluorescence detection. The free L-arginine content in the natto freeze-dried powder was 0.23 g/100 g, while that in the natto oligopeptide powder was 5.5 g/100 g, indicating effective cleavage of peptide bonds around arginine in the natto freeze-dried powder and co-elution of free L-arginine with antithrombotic peptide components during strong cation-exchange chromatography. The free L-arginine content in the natto oligopeptide powder was significantly higher than that in the natto freeze-dried powder.

### Experimental Example 2: Peptide Analysis

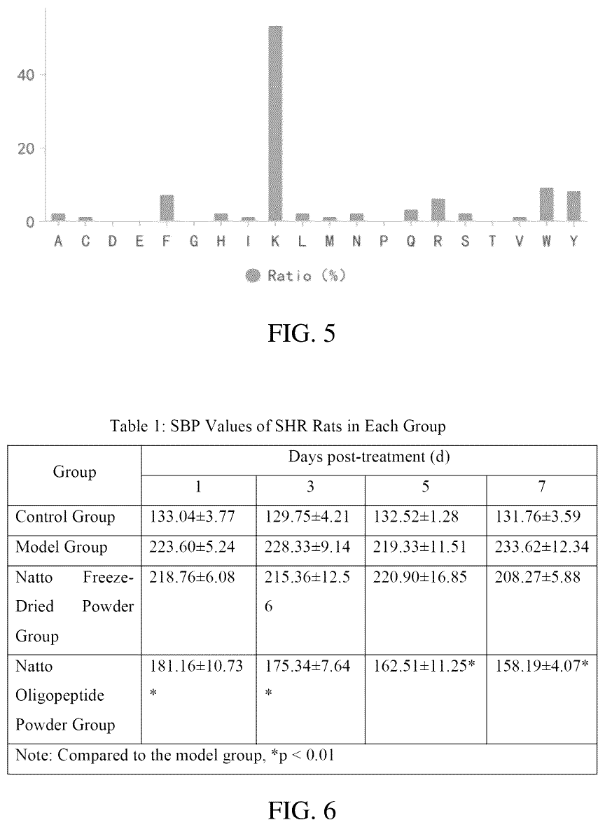

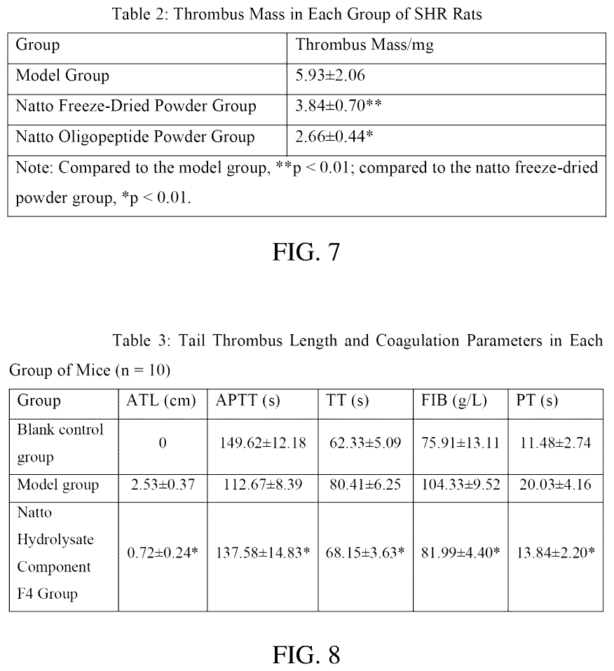

The peptide sequences in the natto oligopeptide powder were analyzed using the same method as in step S2 of Example 1. The proportions of N-terminal and C-terminal amino acid residues are shown in FIGS. 3 and 4. The N-terminal amino acid residues in the natto oligopeptide powder were primarily glycine, while the C-terminal amino acid residues were mainly lysine, with some arginine, tyrosine, and tryptophan, consistent with the targeted enzymatic hydrolysis effect.

### Experimental Example 3: Chronic Thrombosis Model in Spontaneously Hypertensive Rats (SHR)

The natto freeze-dried powder and natto oligopeptide powder were prepared into 1 g/ml pastes using deionized water. Eight WKY rats served as the control group, while 24 SHR rats were randomly divided into three groups of eight each: model group, natto freeze-dried powder group (10 g/kg·d), and natto oligopeptide powder high-dose group (10 g/kg·d). The SHR rats were anesthetized with an intraperitoneal injection of 3% sodium pentobarbital (2 mL/kg), and a 2 cm midline incision was made in the neck to expose the bilateral carotid arteries. A custom plastic pad was placed under the carotid arteries to protect surrounding tissues. A filter paper ring soaked with 2 μL of 50% FeCl₃ solution was applied to the artery for 50 minutes to induce non-occlusive arterial thrombosis, occupying ⅓ to ½ of the vascular lumen. The area was rinsed with physiological saline, and penicillin was applied to prevent infection before suturing. The sham surgery group received a filter paper ring soaked with 2 μL of physiological saline for 50 minutes before suturing. Starting the day after modeling, the treatment groups were administered the respective formulations by gavage, while the control and model groups received an equivalent volume of physiological saline for one week. Blood pressure was measured on days 1, 3, 5, and 7 using a sphygmomanometer, with five measurements per rat, and the average systolic blood pressure (SBP) was recorded. On day 8, the rats were anesthetized with 20% urethane, and the thrombosed carotid artery segment was excised, blotted dry, and weighed using an electronic analytical balance.

Results: As shown in Table 1, the SBP values of SHR rats were significantly higher than those of the control group, and the presence of non-occlusive carotid artery thrombosis further elevated blood pressure compared to normal SHR rats. The SHR rats treated with natto freeze-dried powder showed no significant difference in blood pressure compared to the model group, while those treated with natto oligopeptide powder exhibited a gradual reduction in blood pressure over one week, showing a significant difference from the model group. This indicates that the natto oligopeptide powder has a good vasodilatory and antihypertensive effect. Additionally, the thrombus mass in the carotid arteries of SHR rats was significantly lower in both the natto freeze-dried powder and natto oligopeptide powder groups compared to the model group (p<0.01). The natto oligopeptide powder group, due to the antiplatelet aggregation and anticoagulant effects of the antithrombotic peptide component F4, further reduced thrombosis caused by endothelial cell damage on top of the fibrinolytic effect of the nattokinase component F1, resulting in superior antithrombotic efficacy compared to the natto freeze-dried powder group (p<0.01).

TABLE 1
SBP Values of SHR Rats in Each Group

| | Days post-treatment (d) | | | |
|---|---|---|---|---|
| Group | 1 | 3 | 5 | 7 |
| Control Group | 133.04 ± 3.77 | 129.75 ± 4.21 | 132.52 ± 1.28 | 131.76 ± 3.59 |
| Model Group | 223.60 ± 5.24 | 228.33 ± 9.14 | 219.33 ± 11.51 | 233.62 ± 12.34 |
| Natto Freeze- | 218.76 ± 6.08 | 215.36 ± 12.56 | 220.90 ± 16.85 | 208.27 ± 5.88 |
| Dried Powder | | | | |
| Group | | | | |
| Natto | 181.16 ± 10.73* | 175.34 ± 7.64* | 162.51 ± 11.25* | 158.19 ± 4.07* |
| Oligopeptide | | | | |
| Powder Group | | | | |

Note:
Compared to the model group, *p < 0.01

TABLE 2
Thrombus Mass in Each Group of SHR Rats

| Group | Thrombus Mass/mg |
|---|---|
| Model Group | 5.93 ± 2.06 |
| Natto Freeze-Dried Powder Group | 3.84 ± 0.70** |
| Natto Oligopeptide Powder Group | 2.66 ± 0.44* |

Note:
Compared to the model group, **p < 0.01; compared to the natto freeze-dried powder group, *p < 0.01.

The present invention discloses a method for preparing natto oligopeptides and their application in thrombolysis and blood pressure reduction. The method uses natto as the raw material, first separating the nattokinase component through ultrafiltration. The remaining components are subjected to peptide sequence detection to statistically analyze the proportions of amino acid residues at the N-terminus of arginine. A composite protease formulation targeting this site is designed to enzymatically hydrolyze the remaining components, resulting in a hydrolysate with high L-arginine content and further release of natto antithrombotic peptides and ACE inhibitory peptides. The nattokinase component is then mixed with the hydrolysate, and the mixture is freeze-dried to obtain natto oligopeptide powder. This preparation method preserves the thrombolytic activity of nattokinase in natto while enhancing the thrombolytic effect of the final natto oligopeptide product. Combined with the vasoregulatory effects of L-arginine, this method holds broad prospects in applications such as thrombolysis, blood pressure reduction, and alleviation of thrombotic cardiovascular diseases.

### Example 4: Antithrombotic Effect of Natto Hydrolysate Component F4

Thirty ICR male mice were randomly divided into three groups: blank control group, model group, and antithrombotic group, with 10 mice in each group. The blank group received daily intraperitoneal injections of physiological saline solution (0.01 mL/g body weight), the model group received intraperitoneal injections of 0.20% carrageenan physiological saline solution (0.01 mL/g body weight), and the antithrombotic group was orally administered natto hydrolysate component F4 (20 mg/g body weight) at the same dose of carrageenan injection. After 10 days, the length of black tails in each group was observed and recorded, and blood was collected for semi-automatic coagulation analyzer testing of four coagulation parameters. The results, as shown in Table 3, indicate that the model group developed thrombotic black tails, while the tail thrombus length in the natto hydrolysate component F4 intervention group was significantly shorter compared to the model group. Additionally, the four coagulation parameters showed significant improvement compared to the model group, demonstrating that natto hydrolysate component F4 possesses certain antithrombotic activity.

TABLE 3
Tail Thrombus Length and Coagulation Parameters in Each Group of Mice (n = 10)

| Group | ATL (cm) | APTT (s) | TT (s) | FIB (g/L) | PT (s) |
|---|---|---|---|---|---|
| Blank | 0 | 149.62 ± 12.18 | 62.33 ± 5.09 | 75.91 ± 13.11 | 11.48 ± 2.74 |
| control | | | | | |
| group | | | | | |
| Model | 2.53 ± 0.37 | 112.67 ± 8.39 | 80.41 ± 6.25 | 104.33 ± 9.52 | 20.03 ± 4.16 |
| group | | | | | |
| Natto | 0.72 ± 0.24* | 137.58 ± 14.83* | 68.15 ± 3.63* | 81.99 ± 4.40* | 13.84 ± 2.20* |
| Hydrolysate | | | | | |
| Component | | | | | |
| F4 Group | | | | | |

ATL: Average Thrombus Length;
APTT: Activated Partial Thromboplastin Time;
TT: Thrombin Time;
FIB: Fibrinogen;
PT: Prothrombin Time
Compared to the model group, *p < 0.01

The method for preparing natto oligopeptides according to the present invention uses natto as the raw material. Initially, the nattokinase component is isolated through ultrafiltration. The remaining components are subjected to peptide sequence detection to statistically analyze the proportions of amino acid residues at the N-terminus of arginine. A composite protease formulation targeting this site is designed to enzymatically hydrolyze the remaining components, resulting in a hydrolysate with high L-arginine content and further release of natto antithrombotic peptides and ACE inhibitory peptides. The nattokinase component is then mixed with the hydrolysate, and the mixture is freeze-dried to obtain natto oligopeptide powder. This preparation method preserves the thrombolytic activity of nattokinase in natto while enhancing the thrombolytic effect of the final natto oligopeptide product. Combined with the vasoregulatory effects of L-arginine, this method holds broad prospects in applications such as thrombolysis, blood pressure reduction, and alleviation of thrombotic cardiovascular diseases.

Finally, it should be noted that the above examples are only a few specific embodiments of the present invention. All derivatives directly derived or conceived by those skilled in the art based on the content disclosed in the present invention should be considered within the scope of protection of the present invention.

1. A method for preparing natto-derived antithrombotic oligopeptides, comprising:
providing a natto powder solution;
filtering a natto powder solution through a 29000 Da ultrafiltration membrane to obtain a first filtrate with a molecular weight less than 29000 Da and a first retentate with a molecular weight greater than 29000 Da;
filtering the first filtrate through a 26000 Da ultrafiltration membrane to obtain a second filtrate with a molecular weight less than 26000 Da and a second retentate with a molecular weight greater than 26000 Da and less than 29000 Da; wherein the first retentate and the second filtrate are combined as a natto component solution, and the second retentate is a nattokinase component solution;
retrieving and determining characteristics of peptides associated with natto's antithrombotic function from a bioactive peptide database, wherein the characteristics comprise: molecular weight <2000 Da, N-terminal glycine (G), C-terminal lysine (K) or arginine (R), and net charge ±2;
adding trypsin to the natto component solution and enzymatically hydrolyzing to obtain a natto small peptide solution;
sequencing peptides in the natto small peptide solution, and statistically analyzing types and quantities of amino acid residues preceding glycine (G) and arginine (R) in the natto small peptide solution;
selecting cleavage sites based on statistical ranking of the residues, wherein the cleavage sites include: tyrosine-glycine peptide bonds, phenylalanine-glycine peptide bonds, tyrosine-arginine peptide bonds, lysine carboxyl-terminal peptide bonds, and arginine carboxyl-terminal peptide bonds;
enzymatically cleaving the cleavage sites using a composite protease comprising trypsin and chymotrypsin in a ratio of 1:(1-3), under conditions of: temperature 37-45° C., pH 7.5-8.5, and hydrolysis time 3-6 hours, thereby obtaining a cleaved peptide solution;
filtering the cleaved peptide solution through a nanofiltration membrane with a molecular weight cutoff of 2000 Da to collect a solution of peptides <2000 Da;
purifying the <2000 Da peptide solution via ion-exchange chromatography to collect peptides with a net charge of ±2, thereby obtaining a first oligopeptide solution; and
mixing the ±2 net charge peptide solution with the nattokinase component solution to obtain a second oligopeptide solution, thereby obtaining the natto-derived antithrombotic oligopeptides; and
wherein the selecting cleavage sites based on statistical ranking of the residues further comprises:
sorting amino acid residues preceding glycine (G) by quantity and cleaving peptide bonds of the most abundant residues to obtain N-terminal glycine (G) peptides;
sorting amino acid residues preceding arginine (R) by quantity and cleaving peptide bonds of the most abundant residues to obtain N-terminal arginine (R) peptides;
cleaving lysine carboxyl-terminal peptide bonds to obtain C-terminal lysine (K) peptides; and
cleaving arginine carboxyl-terminal peptide bonds of non-N-terminal arginine (R) peptides to obtain C-terminal arginine (R) peptides, and cleaving N-terminal arginine (R) peptides to release free L-arginine.

2. The method according to claim 1, wherein:
filtering the cleaved peptide solution comprises using a 2000 Da nanofiltration membrane to obtain a third filtrate, wherein the third filtrate is the <2000 Da peptide solution;
purifying the <2000 Da peptide solution comprises the ion-exchange chromatography.

3. The method according to claim 2, wherein the ion-exchange chromatography utilizes a strong cation-exchange column.

4. The method according to claim 3, wherein:
the strong cation-exchange column is a sepharose high performance column;
after loading, the column is equilibrated with 20 mmol/L phosphate buffer (pH 6.0) and eluted with a 0-1 M NaCl gradient over 0-40 minutes, collecting eluate from 11-19 minutes to obtain a ±2 net charge peptide solution as the first oligopeptide solution.

5. The method according to claim 1, wherein the natto is prepared from soybeans.

6. The method according to claim 1, further comprising freeze-drying the first oligopeptide solution to obtain natto oligopeptide powder.

7. The method according to claim 1, further comprising freeze-drying the second oligopeptide solution to obtain natto oligopeptide powder.
